(19) United States
(12) Patent Application Publication
(10) Pub. No.: US 2008/0195009 A1
(43) Pub. Date: Aug. 14, 2008

(54) WATERPROOF LIMB PROTECTOR

(76) Inventor: Steve M. Satkowiak, Escondido, CA (US)

Correspondence Address: GORDON & REES LLP, 101 WEST BROADWAY, SUITE 1600, SAN DIEGO, CA, 92101, US

(21) Appl. No.: 12/047,208

(22) Filed: Mar. 12, 2008

Related U.S. Application Data
(63) Continuation-in-part of application No. 11/406,225, filed on Apr. 17, 2006, now abandoned.
(60) Provisional application No. 60/672,451, filed on Apr. 18, 2005.

(51) Int. Cl. A61F 5/00

(52) U.S. Cl. 602/3

(57) ABSTRACT

The present invention relates to a waterproof limb protector for preventing moisture from in contact with an affected area of an injured limb. The waterproof limb protector comprises an elongated neoprene sheath with an open end for receiving the limb in need of protection, and an adjustable neoprene sealing strap with a fixed end attached adjacent to the open end of the sheath. The sealing strap further comprises a VELCRO® fastener with one fastener member attached to the distal free of the sealing strap and the other fastener member to the middle section of the sealing strap. The two fastener members are attached on the opposite side of the sealing strap. To secure the sheath to an injured limb, the sealing strap is wrapped around the circumference of the open end as such that the two VELCRO® fastener members are in contact to provide an effective but comfortable watertight seal around the open end. Alternatively, the sealing strap may further comprise a VELCRO® hook fastener on the inside surface of the free end, and a looped outside surface that is matable with the hook fastener for facilitated one-handed engagement.

### CROSS REFERENCE TO RELATED APPLICATION

This application is a Continuation In Part of U.S. patent application Ser. No. 11/406,225 filed Apr. 17, 2006, which claims the benefit of U.S. Provisional Application No. 60/672,451, which was filed on Apr. 18, 2005.

### TECHNICAL FIELD

The present invention relates to a waterproof limb protector for preventing moisture from contacting an affected area of an injured limb.

### BACKGROUND OF THE INVENTION

Every year, thousands of Americans suffer from broken bones. Whether twisting an ankle after tripping down a flight of stairs, fracturing a leg in an unfortunate skiing accident, or falling off of a bicycle and breaking one's arm, fractures are a painful injury that are typically slow to heal. The most common treatment for a fracture is setting the broken bones into proper alignment, and then wrapping the injured area in a plaster or resin-coated cast. The purpose of a cast is to hold the broken bones in place as they heal, restricting movement and ensuring that it heals properly, a process that can take anywhere from a few days to several weeks, depending upon the severity of the fracture.

While wearing a cast is an important part of the healing process, they can be a nuisance. Because casts can soften if they become wet and because this moisture can promote bacterial infection, most orthopedists give their patients strict instructions to keep their casts dry at all times. Although keeping a cast dry through normal daily activities is usually not a problem, keeping it dry while taking a bath or a shower can be nearly impossible. Trying to properly bathe while holding an arm out of the shower or tub can be awkward, difficult, and in many cases, quite painful. For those with a broken leg or ankle, the task is exponentially more daunting. Many people attempt to solve this problem by wrapping their casts in a plastic bag or plastic-wrap and taking their bath or shower quickly. Unfortunately, this method is often ineffective as water can leak in through the bag's opening, soaking the cast and rendering it useless.

Accordingly, there is a need for a limb protector that would protect an injured limb from moisture and wetness due to showing or bathing. The present invention provides a limb protector which is a reusable sheath that fits securely around an injured limb for protecting it from water damage.

### SUMMARY OF THE INVENTION

In accordance with the present invention, a waterproof limb protector is provided for preventing moisture from contacting an affected area of an injured limb. The waterproof limb protector comprises an elongated neoprene sheath with a closed end and an open end for receiving the limb in need of protection, and a stretchable neoprene sealing strap with three segments, a fixed end, an intermediate segment, and a distal free end, the fixed end being attached adjacent to the open end of the sheath. The closed end may have a general shape of sack with a tapered end, which can be universally applicable to both arms and legs. The closed end may also be specially dimensioned and adapted to receive a hand or foot.

The sealing strap further comprises a VELCRO® fastener with two or more matable members, having one fastener member attached to the distal free end on one surface of the sealing strap and the other fastener member to its intermediate segment on the opposite surface. To secure the sheath to an injured limb and to provide an effective but comfortable watertight seal around the periphery of the open end, the stretchable sealing strap is wrapped around the circumference of the open end as such that the two matable VELCRO® fastener members are overlapped substantially.

The use of the waterproof limb protector of the present invention is simple and straightforward, including the steps of extending an injured limb into the sheath and securing the sheath to the affected limb by wrapping the sealing strap around the circumference of the open end and locking the sealing strap via the two matable VELCRO® fastener members.

In one embodiment which is particularly well adapted for one-handed engagement, the waterproof limb protector comprises: a neoprene elongated sheath with a closed end and an open end for receiving an injured limb, wherein the elongated sheath further comprises an inside surface and an outside surface; and a stretchable neoprene sealing strap that has; an inside surface and an outside surface, wherein the outside surface is looped and removably matable with a hooked surface of a hook-and-loop fastener system; a free end and a fixed end, wherein the fixed end is attached via the inside surface to the outside surface adjacent to the open end; at least one hooked fastener attached to the inside surface of the free end, wherein the hooked fastener is removably matable with the looped outside surface; wherein the stretchable sealing strap is configured to wrap around the open end of the elongated sheath such that the hooked fastener fixably mates with the looped outside surface to form a watertight seal around the open end of the elongated sheath about the limb.

### BRIEF DESCRIPTION OF THE DRAWINGS

FIG. 1 is a perspective view of a one-fits-all waterproof limb protector with a single open end.

FIG. 2 is a perspective view of a waterproof limb protector with two open ends for elbow or knee.

FIG. 3 is a protection is a perspective view of a waterproof limb protector with a closed end in a hand shape for protecting hand and/or arm protections.

FIG. 4 is a perspective view of a waterproof limb protector with a closed end in a foot shape for foot and/or leg protections.

FIG. 5 show a single piece flat sheet (A) or two-piece flat sheets (B) to be manufactured into a waterproof limb protector.

### DETAILED DESCRIPTION OF THE INVENTION

The present invention relates to a waterproof limb protector 1 for preventing moisture from contacting an affected area of an injured limb. The term “limb”, as used herein, refers to an arm, forearm, elbow, wrist, hand, finger, leg, knee, ankle, foot, or toe which might be protected by a medical dressing. The term “medical dressing” may be understood to comprise casts, splints, bandages, stitches, surgical staples, or any other protective material covering or support for an injured or wounded limb.

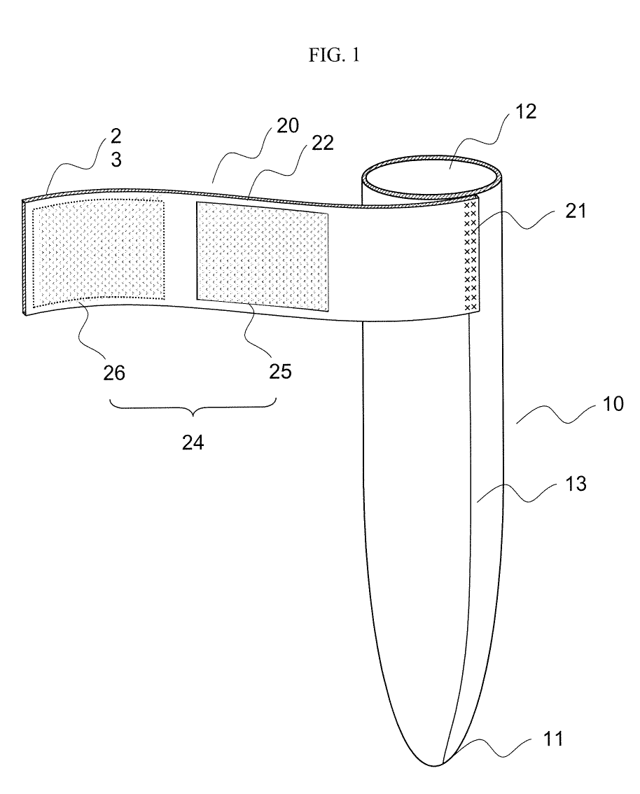

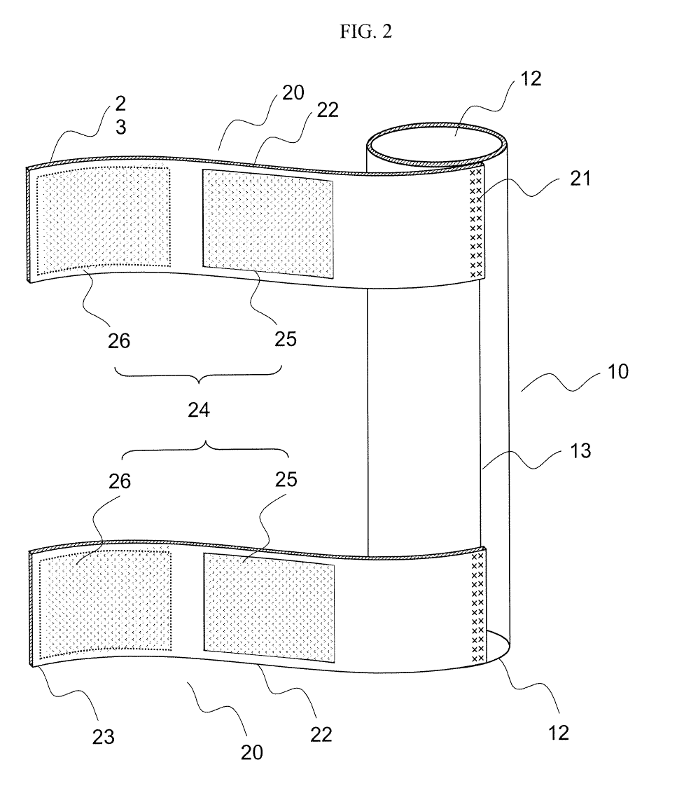

The waterproof limb protector 1 of the present invention comprises a sheath 10 and a stretchable sealing strap 20. The sheath 10 generally has an elongated tubular shape with a closed end 11 and an open end 12 for receiving an injured limb (FIG. 1). Alternatively, the sheath 10 may also have two open ends, each open end 12 having one sealing strap 20 attached close to that open end 12 (FIG. 2). The sheath 10 with two open ends may be used to protect an injured elbow or knee. In spite of one or two open ends 12 on the sheath 10, each open end 12 must be capable of forming a watertight seal against an injured limb once the limb has been inserted into the sheath 10.

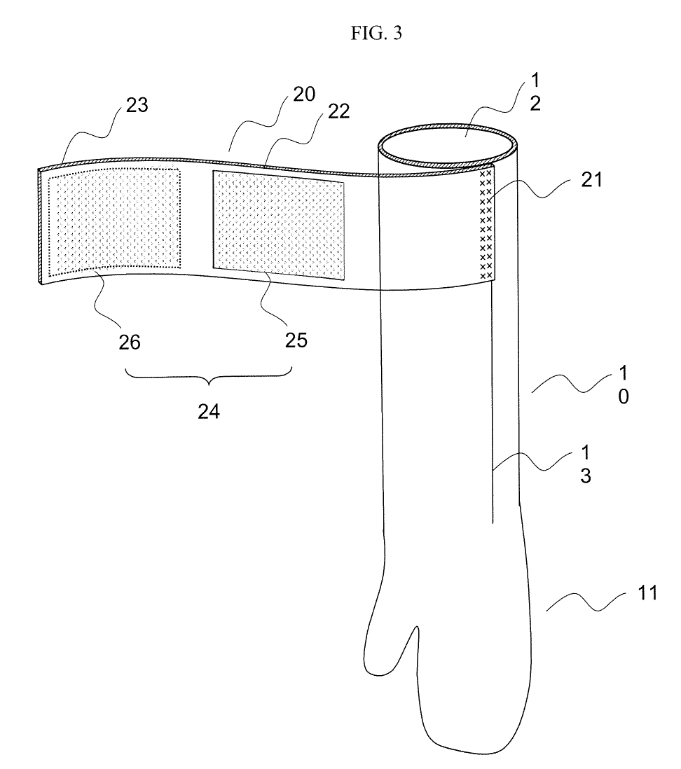

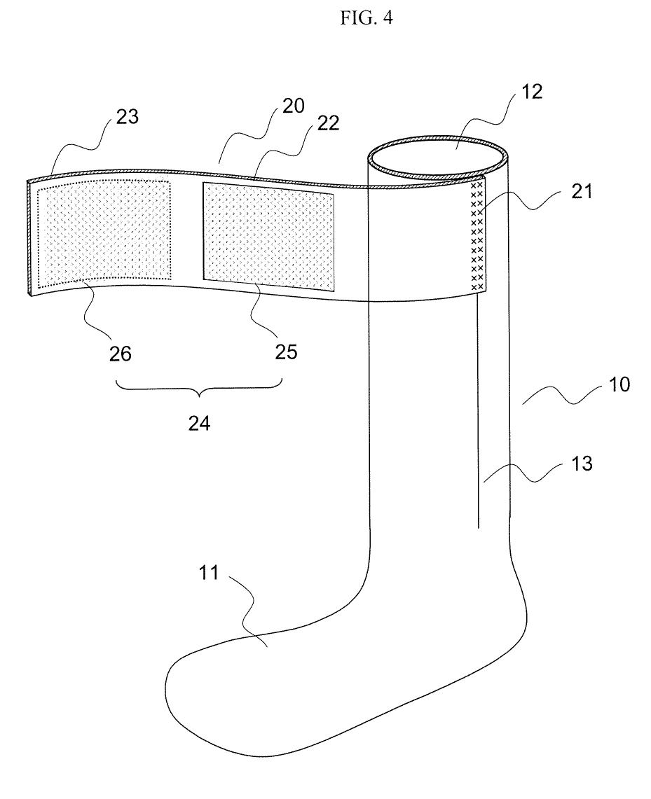

The closed end 11 may have a general shape of sack with a tapered end, which can be universally applicable to both arms and legs. The closed end 11 may also have a shape specifically for an arm or leg to provide additional comforts during use. For example, the closed end 11 of the sheath 10 may be in a mitten shape with a thumb compartment for use over an injured arm (FIG. 3). The closed end 11 may also be dimensioned and adapted to receive a foot for use over an injured leg (FIG. 4).

The open end 12 for receiving an injured limb may have various diameters to accommodate different limb sizes. In general, the open end 12 has a circumference slightly larger than the circumference of an injured limb, so that the sheath 10 may loosely fit over an injured limb to ease insertion of the injured limb into the open end 12. In the embodiments of the present invention, the open end 12 may have a circumference ranging from about 5 to about 30 inches, from about 5 to about 20 inches, from about 5 to about 15 inches, or from about 5 to about 10 inches. For instance, the waterproof limb protector 1 to fit over an average casted adult arm would have an open end 12 with a circumference of approximately 6 to 9 inches. In general, the sheath 10 has a main body with the same diameter as the open end as described above.

The sheath 10 may be manufactured in various lengths and styles to sufficiently cover a full arm, wrist, full leg, or foot. In general, the sheath 10 is designed to receive an injured adult arm or leg with an average size. Preferably, the sheath 10 can be provided as a one-size-fits-all standard for adults. For instance, the waterproof limb protector 1 to fit over an average casted adult arm would have a length of approximately 24 to 32 inches. However, the sheath 10 can also be easily made available in smaller sizes appropriate for children. In the embodiments of the present invention, the sheath 10 may have a length ranging from about 6 to about 50 inches, from about 7 to about 40 inches, from about 8 to about 30 inches, or from about 10 to about 25 inches.

The sheath 10 of the present invention is composed of resilient and elastic and waterproof plastic material, particularly lightweight heavy-gauge plastic sheet material, such as a neoprene sheet, which provides both durability and elasticity. The plastic sheet may have a thickness of no less than about 0.1 mm, no less than about 1 mm, no less than about 1.5 mm, no less than about 2 mm, no less than about 3 mm, no less than about 4 mm, no less than about 5 mm, no less than about 6 mm, or no less than about 10 mm. To be durable and reusable, the plastic sheet should be thick enough to endure stretching and pulling forces during repetitive usages without tearing. In certain embodiments of the present invention, the plastic sheet may have a thickness of about 1 mm, about 1.5 mm, about 2 mm, about 3 mm, about 4 mm, about 5 mm, or about 6 mm. The plastic material may also contain a lining to further enhance physical characteristics, such as elasticity and durability. For example, neoprene may be lined with a nylon fabric to strengthen it and make it easy to put on and take off. The neoprene material may also contain spandex in addition to neoprene to allow the sheath 10 to further increase its elasticity. Preferably, the neoprene material may contain from about 15 to about 20% spandex. Such neoprene material is also known as superflex, which counteracts neoprene's tendency to shrink with age, and also allows for some changes in the wearer's size without making the sheath 10 uncomfortable. The plastic material for use in the present invention may be transparent, translucent or opaque and is not limited to any particular color or decorative pattern.

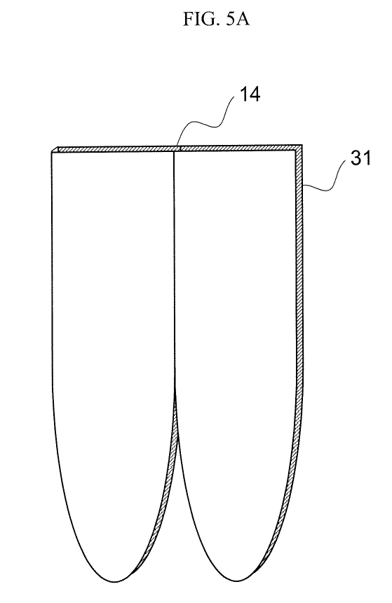

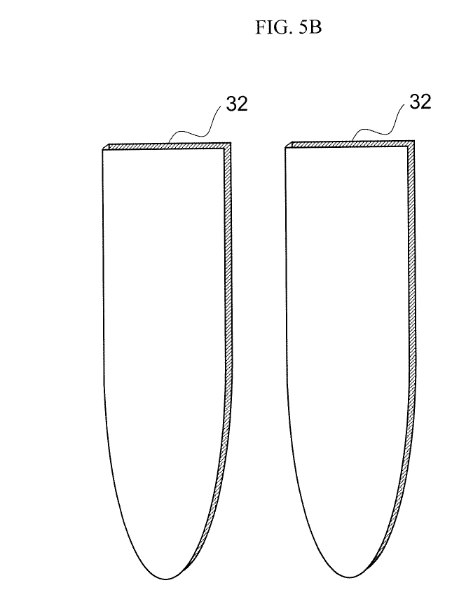

The sheath 10 of the present invention may be molded as a single seamless piece. The sheath 10 may also be fabricated using a single piece construction as shown in FIG. 5A. For example, the neoprene sheet is cut to a width which is twice that of the completed sheath 10. The neoprene sheet 31 is then folded over along the folding line 14 and the edges are first stitched together without overlapping edges to form a single longitudinal seam 13. The seam 13 may further be sealed with a water-resistant chemical, such as a glue or a sealing tape, to ensure that no water will leak through the seam 13 while the limb protector 1 is in use. The seam 13 may be sealed on the interior surface, the exterior surface or both.

Alternatively, the sheath 10 may be fabricated using a two-piece construction. As shown in FIG. 5B, for example, the neoprene sheet is cut into two pieces 32, each having the same width as that of the completed sheath 10. The edges are first stitched together without overlapping to form two longitudinal seams 13. Similarly, the seams 13 may further be sealed with a water-resistance chemical to ensure that no water will leak through the seams 13 while the limb protector 1 is in use.

The sealing strap 20 of the limb protector 1, as shown in FIG. 1, is a rectangular elongated band with three segments: a fixed end 21, an intermediate segment 22, and a distal free end 23. The fixed end 21 of the sealing strap 20 is attached to the sheath 10 close to the open end 12. As such, the sheath 10 can be easily secured by wrapping the sealing strap 20 around the circumference of the open end 12 using a single hand. The sealing strap 20 may be attached in various ways. The top longitudinal edge of the sealing strap 20 may be slightly above, slightly below, or at the same level as the top edge of the open end 12. Preferably, the top longitudinal edge of the sealing strap 20 is parallel to the top edge of the open end 12.

The sealing strap 20 may permanently attached to the sheath 10 through a variety of methods well known in the art, including stitching and bonding with a chemical adhesive such as a glue. In the embodiments of the present invention, the sealing strap 20 is stitched with one of its two surfaces to the exterior surface of the sheath 10. Alternatively, the sealing strap 20 may be temperately attached to the sheath 10 by using a detachable fastening mechanism, such as VELCRO® strips. The sealing strap 20 may be made from the same or similar material to the sheath 10 as described above, which is elastic or stretchable to insure that effective but comfortable watertight seal is provided between an injured limb and the open end 12 of the sheath 10.

The sealing strap 20 of the present invention is of a size and elasticity as to apply sufficient elastic force for sealing the sheath 10 to an injured limb, yet without being too tight to affect blood circulation. The sealing strap 20 may have a width of from about 0.5 to about 10 inches, from about 1 to about 8 inches, from about 1.5 to about 6 inches, from about 1.5 to about 5 inches, about 2 inches, about 3 inches, about 4 inches, about 5 inches, or about 6 inches. The sealing strap 10 may have a length which is greater than the circumference of the open end 12 of the sheath 10. Preferably, the sealing strap 20 is at least 1.1, at least 1.2, at least 1.3, at least 1.4, at least 1.5, at least 1.6, at least 1.7, at least 1.8, at least 1.9, or at least 2 times of the circumference of the open end 12. To fit over an average casted adult arm, the sealing strap 20 would have a length of approximately 7 to 18 inches.

The sealing strap 20 further comprises a fastener 24 with two matable members for securing the limb protector 1 around the limb that is to be protected and providing a watertight seal around the periphery of the open end 20 when the limb protector 1 is in use. The first matable member 25 is attached to the intermediate segment 22 on one side, preferably opposite to the surface where the sheath 10 is attached. The second matable member 26 is attached to the distal free end 23, preferably on the same surface where the sheath 10 is attached. When the sealing strap 20 is wrapped around the open end 12, the fastener member 26 on the distal free end 23 will be engaged with the second member 25 on the intermediate segment 22 to securely lock the sealing strap 20 in position. The fastener 24 may be a hook-and-loop fastener with one matable member having hooks and the other having loops. One example for such hook-and-loop fastener is VELCRO®. One member of the VELCRO® fastener may be attached to the intermediate segment 22 whereas the other member may be attached to the distal free end 23. Each VELCRO® member has a sufficient length along the sealing strip 20 as such that the two members are able to have sufficient overlapping area when wrapped around an injured limb, to provide an effective watertight seal for users with different limb sizes. Each fastener member (25 or 26) may independently have a width slight small than the width of the sealing strap 20 and a length ranging from about 2 inches up to the length of the sealing strap 20, from about 2 to about 15 inches, from about 4 to about 12 inches, or from about 5 to about 10 inches. It would be preferred that the lengths of the two matable fastener members is matched.

In an alternative embodiment to the one just described, instead of having a VELCRO® type hook-and-loop fastener system with matable fastener members, such as the rectangular-shaped members depicted in FIG. 1 as 25 and 26, a different configuration can be used to facilitate one-handed engagement. More particularly, the outside surface of the sealing strap 20 can be constructed of a “Velcro-sensitive material”, which means that the material is capable of being matably engaged with a standard Velcro-type hook member. Since the standard Velcro-type hook and loop members are usually not stretchy, this limits the amount of surface area rendered inelastic by attachment of the matable fastener members. For example, as shown in FIG. 1, the two fastener members 25 and 26 take up nearly one third of the surface area of the sealing strap 20, rendering it relatively unelastic, since the strap won't readily stretch between the left and right sides of the attached fastener members. In contrast, if only one fastener member is used, such as 26, and it is a Velcro hook-type fastener attached to the inside surface, by constructing the entire sealing strap out of a Velcro-sensitive material on its outside surface, the stretchability of the strap is better maintained.

In addition to the enhanced elasticity of a strap with only one Velcro-type fastener one surface that mates with a Velcro-sensitive material on the other surface, alignment of the overlapping free end of the strap is less critical. For example, using standard Velcro-type hook and loop fastening members, alignment of the hook member with the look member is critical to form a secure attachment. However, no horizontal alignment is necessary in the configuration just described, since the hook fastener member will attach anywhere to the Velcro-sensitive material on the outside surface of the sealing strap. This greatly facilitates the ability to put on the device with one hand. Velcro sheets are commercially available with Velcro-sensitive material on one side. As used herein, the term “wherein the outside surface is looped” intends that the outside surface is covered in a Velcro-sensitive material that is matable with the hook side of a Velcro-type hook-and-loop fastener.

Ideally, in either configuration, the fastener members are rectangles, with a wider length along the sealing strap than the width to provide for a more secure attachment relative to circumferential forces when the sealing strap is stretched in the engaged position. Also, the fastener member or members affixed to the sealing strap cover at least 20% of the total surface area of the sealing strap.

The use of the waterproof limb protector 1 of the present invention is very simple and straightforward. First, a person chooses the protector 1 appropriate for the person's injured limb. For example, a person suffering from a broken hand or wrist selects the glove-like wrist version of the limb protector 1 of the present invention (FIG. 3), whereas a person with a broken ankle selects the foot cast version (FIG. 4). Donning the limb protector 1 as they would any other glove, stocking, or sock, the wearers simply pull the durable limb protector 1 up over an injured limb and wrap the elastic sealing strap 20 around the periphery of the open end 12 to conform to the shape of their arms, elbows, ankles, or legs. Adjusting the VELCRO® sealing strap 20 for comfort and security, the wearer can then take a shower or enjoy a hot bath, ensured that the durable neoprene construction of the waterproof limb protector 1 keeps the affected limb dry, while the elastic sealing strap 20 provides an effective barrier between the water and the protected area. After bathing was complete, the wearer simply removes the limb protector 1, hanging it on a towel bar or storing it in a closet until needed again.

The waterproof limb protector 1 of the present invention provides many benefits and advantages. The limb protector 1 allows those suffering from a broken limb to shower or bathe without the worry of getting their cast wet. Providing a watertight barrier between the wearer's cast and the shower or bath water, the limb protector 1 effectively prevents moisture from damaging the cast, which can result in a longer recovery period or possible infections. The waterproof limb protector 1 is also suitable for wear by those who are recuperating from an external injury to the arm, leg, hand, or foot. Keeping stitches or surgical scars free from moisture during bath time, the limb protector 1 proves a valuable aid in a speedy recovery for a variety of topical ailments. Ordinarily, the waterproof limb protector 1 is used for only a short interval of time permitting the user to bathe or shower. However, the waterproof limb protector 1 of the present invention is also suitable to use for an extended period of time, such as in inclement weather or in a sport activity, without significantly affecting blood circulation.

One skilled in the art will understand that various modifications may be made to the above embodiments which are still within the scope and spirit of the invention described herein. For example, the sealing strap 20 can be attached with an angle between the top edges of the sealing strap 20 and the open end 12. Therefore, the above description should not be construed as limiting but merely as exemplifications of the preferred embodiments of the invention.

The examples set forth above are provided to give those of ordinary skill in the art with a complete disclosure and description of how to make and use the preferred embodiments of the compositions, and are not intended to limit the scope of what the inventors regard as their invention. Modifications of the above-described modes for carrying out the invention that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A waterproof limb protector comprising:
a neoprene elongated sheath with a closed end and an open end for receiving an injured limb, wherein the elongated sheath further comprises an inside surface and an outside surface; and
a stretchable neoprene sealing strap comprising;
an inside surface and an outside surface, wherein the outside surface is looped and removably matable with a hooked surface of a hook-and-loop fastener system;
a free end and a fixed end, wherein the fixed end is attached via the inside surface to the outside surface adjacent to the open end;
at least one hooked fastener attached to the inside surface of the free end, wherein the hooked fastener is removably matable with the looped outside surface;
wherein the stretchable sealing strap is configured to wrap around the open end of the elongated sheath such that that the hooked fastener fixably mates with the looped outside surface to form a watertight seal around the open end of the elongated sheath about the limb.

2. The waterproof limb protector of claim 1, wherein the neoprene elongated sheath is dimensioned to receive an injured limb.

3. The waterproof limb protector of claim 1, wherein the neoprene elongated sheath is made of a first neoprene sheet material.

4. The waterproof limb protector of claim 3, wherein the first neoprene sheet material has a thickness of more than 0.1 mm.

5. The waterproof limb protector of claim 3, wherein the first neoprene sheet material has a thickness of about 1 mm to 6 mm.

6. The waterproof limb protector of claim 3, wherein the stretchable neoprene sealing strap is made of a second neoprene sheet material, wherein the second neoprene sheet material is more elastic than the first neoprene sheet material.
